(19) United States
(12) Patent Application Publication
(10) Pub. No.: US 2025/0160540 A1
(43) Pub. Date: May 22, 2025

(54) RECLINING DEVICE FOR THE STABLE LATERAL POSITIONING OF A CHILD

(71) Applicant: Llevanto GmbH, Berlin (DE)

(72) Inventors: Katharina Schroth, Berlin (DE); Caspar Schroth, Berlin (DE)

(73) Assignee: Llevanto GmbH, Berlin (DE)

(21) Appl. No.: 18/832,601

(22) PCT Filed: Jan. 26, 2023
(86) PCT No.: PCT/EP2023/051923

§ 371 Date: Jul. 24, 2024

(30) Foreign Application Priority Data
Jan. 28, 2022 (EP) 22153850.7

(51) Int. Cl. A47D 15/00; A61F 5/37

(52) U.S. Cl. CPC A47D 15/008; A61F 5/3707

(57) ABSTRACT

A reclining device (1) which, due to its shape, enables a child to be placed in a stable lateral position is provided. The device has a shape with a central region (3), and at least one head region (2) and/or a foot region (4), the central region has a smaller diameter than the head region (2) and the foot region (4). In some versions of the device, the smaller diameter of the central region (3) and/or the at least one padded insert (6) in the central region (3) is designed to prevent a child from adopting a prone or supine position. In further embodiments, the reclining device (1) includes at least one padded insert (6) which can be attached to at least one inner wall (3a) in the central region (3) in order to reduce the diameter (3b) of the central region (3).

### DESCRIPTION

The invention relates to a lying device (1) whose shape enables stable lateral positioning of a child, wherein the shape comprises a central region (3), and at least one head region (2) and/or a foot region (4), wherein the central region has a smaller diameter than the head region (2) and the foot region (4). In preferred embodiments, the smaller diameter of the central region (3) and/or the at least one interior padding (6) in the central region (3) is configured to prevent a prone or supine position of the child. In further embodiments, the lying device (1) comprises at least one interior padding (6) which can be attached to at least one inner wall (3a) in the central region (3) in order to reduce the diameter (3b) of the central region (3).

### BACKGROUND AND STATE OF THE ART

Sleeping in the prone position carries an increased risk of sudden infant death syndrome (SIDS) for infants, e.g., for the first year of life. This is why pediatricians generally recommend sleeping in the supine position for newborns and infants. However, sleeping in the supine position in regular cribs also poses various risks. For example, if a loose blanket is used, it can make it difficult for the child to breathe if it slides over the child's head. Pillows also pose a risk of suffocation in a regular crib and are disadvantageous for the development of the spine.

Infants also sleep significantly worse in the recommended supine position, as they are unable to cope well with the sudden space after 9 months of being ‘swaddled’ (tightly enclosed) in the embryo position in the womb. Due to the lack of boundaries and contact, especially in the middle and front of the baby's body, regular sleep in the supine position is often disturbed by shooting muscle twitches (myoclonus) and motor reflexes. The increased restlessness caused by this favors the formation of vertebrae in the intestines and bloating and thus pain.

In addition, the supine positioning of infants can cause the still malleable (soft) skull of infants to flatten. This flattening can vary in severity: from a purely cosmetic deformation to a pathological flattening of the skull (plagiocephalus), which can also affect the supporting device of the neck/jaw. Treatment consists of either positioning (mild cases), physiotherapy (moderate cases) or frequently painful helmet therapy (severe cases).

Lateral positioning of infants can only be recommended by pediatricians if it is ensured that the infant cannot turn into the prone position (roll-over protection). Pediatricians do not recommend unsupported lateral positioning, as the child can easily roll from the side onto the stomach, which in turn increases the risk of sudden infant death syndrome.

The state of the art only offers various pillow systems for lateral positioning of infants. However, these pillows, some of which are loose, have to be fitted into a crib and may again pose a risk of suffocation. These pillows can also shift in the crib so that the child still rolls into the prone position. These sometimes very large lateral positioning pillow systems also rarely fit into a regular carrycot, which simply prevents lateral positioning of a child during transport or can also hinder the child's breathing due to the pillows slipping in the narrow carrycot.

One example of systems designed to provide cushions for regulating the position of infants is U.S. Pat. No. 5,524,640A. The system described in U.S. Pat. No. 5,524,640A uses a variable plug-in system of cushions, which is suitable for weakened premature or sick infants and is intended to stabilize them, for example for positioning during infusions. However, this device is not suitable for transporting an infant or as a carrying device. In particular, the system according to U.S. Pat. No. 5,524,640A is not suitable for effectively preventing a supine or prone position in healthy or older infants, who have a greater urge to move and strength than sick or premature infants, due to its plug-in padding inserts. In the case of healthy infants, the system according to U.S. Pat. No. 5,524,640A poses the risk of the interior padding being displaced or detached when a healthy infant struggles in the device or holds on to the inserts, so that a stable lateral position cannot be guaranteed.

Carrying systems for infants or toddlers, which are intended to make carrying more comfortable, especially for the person carrying them, are specified in U.S. Pat. No. 6,354,475B1 and WO2014/132030A2, for example. However, these devices are not suitable/designed to secure a stable lateral position of an infant and reliably prevent a supine or prone position. The carrying system according to WO2014/132030A2 is even specially designed for the supine position of a child.

A safe and comfortable solution for the lateral positioning of children, especially newborns and infants, is therefore not possible with the means known from the state of the art.

### SUMMARY OF THE INVENTION

The task of the present invention was to eliminate the disadvantages of the prior art and to provide an improved lying device for children which offers safe and comfortable lateral positioning of newborns and infants in order to avoid the disadvantages of the supine position (poorer sleep, skull deformation) without having to switch to the dangerous prone position and to give the baby the greatest possible comfort with regard to its demands, i.e. better sleep by limiting/holding the baby in the lateral position and relieving the pressure on the back of the head.

The task according to the invention is solved by the features of the independent claims. Preferred embodiments of the invention are described in the dependent claims.

In one aspect, the invention therefore relates to a lying device (1) whose shape enables stable lateral positioning of a child, wherein the shape comprises a central region (3), and at least one head region (2), and/or a foot region (4), wherein the central region has a smaller diameter than the head region (2) and the foot region (4).

In one embodiment of the lying device (1), which enables stable lateral positioning of a child due to its shape, the shape comprises a central region (3), a head region (2), and/or a foot region (4), wherein the central region has a smaller diameter than the head region (2) and the foot region (4).

In some embodiments, the lying device thus comprises either a head region or a foot region, but always a central region (3) whose diameter is smaller than that of the existing head and/or foot region.

In one aspect, the invention therefore relates to a lying device (1) whose shape enables stable lateral positioning of a child, wherein the shape comprises a head region (2), a central region (3) and a foot region (4), wherein the central region has a smaller diameter than the head region (2) and the foot region (4).

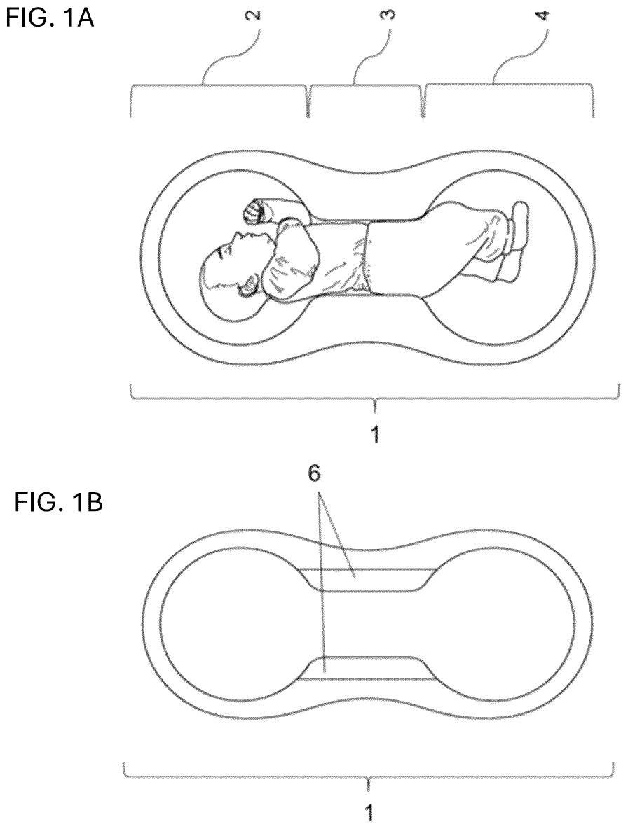

In specific embodiments of the lying device (1), the border or edge of the head and/or foot region is interrupted or not continuous, so that the lying device is “open” or not framed towards the foot region and/or the head of the lying device and the child lying therein. Thus, in some embodiments, the lying device resembles an hourglass (open at the top and/or bottom) (in other words with walls “open” at the head region (in the head direction) and/or in the foot region (in the foot direction)) rather than a closed 8-shape. These specific embodiments allow a very variable adaptation of the lying device according to the invention to the body height of a child and yet a stable lateral positioning. Non-limiting examples of this hourglass shape, which is open at the bottom and top, are shown in FIG. 1 C)-E).

Non-limiting embodiments of this lying device, which in the view of an hourglass is open at the top and/or bottom, may comprise a base and only two side walls, which are designed in such a way that they prevent a child lying in the device from lying in a supine position. In embodiments, the lying device can be designed in such a way that the walls are stably upright, i.e. perpendicular to the base, and allow a child to lie down in it. The skilled person is familiar with modifications of such a lying device and/or its components which enable an upright or stable (vertical) position of the walls.

Special embodiments are also disclosed herewith in which the bottom is very narrow or merely designed as a band/strap/belt, optionally also as an adjustable band/strap/belt, which connects the side walls to each other. In some of these embodiments, the side walls can additionally be stabilized by a strap or belt above/on the upper side of the child lying in the device and preferably secured to the child, so that a lateral position of the child is ensured.

In some embodiments, at least one wall of this “hourglass-shaped” device, which resembles an hourglass open at the top and/or bottom, is shaped such that the diameter of the central region is reduced by the shape of at least one wall itself, so that the device preferably has a “tapered” inner and optionally also outer shape in the central region (see FIG. 1C-D). In embodiments, the inner diameter of the central region can additionally be reduced, for example for very young, small or thin children or infants, by at least one interior padding (e.g. FIG. 1E)

In some embodiments, a device according to the invention, which is designed with “open” /discontinuous walls (in the head direction) in the top view at the head region and/or in the foot region, has at least one straight-cut (lateral) outer wall or two straight-cut, parallel (lateral) outer walls (as shown for an embodiment with closed walls in FIG. 1F). In such embodiments, the inner diameter of the central region can be reduced by at least one interior padding attached to at least one inner wall and/or by a tapered/narrowing design of the inner wall itself to prevent a child from lying on its back and/or stomach.

In some embodiments, the edge has a different height in different areas of the lying device.

In embodiments, the edge of the lying device (1) thus has a different height in the central region (3), head region (2) and/or foot region (4) or is interrupted in the head and/or foot region.

Preferably, the lying device according to the invention is set up to ensure a lateral positioning fixation for children. In embodiments, the child is placed sideways in the lying device (1). The child's torso is fixed by the central region so that the child cannot turn onto its back or stomach. In particular, this ensures protection against rolling forward. In embodiments, the child to be placed sideways in the lying device is fixed at the torso by the central region, and optionally by additional interior padding attached there, while the head and leg sections have freedom of movement.

Surprisingly, in the course of her work in a pediatric practice, the inventor has repeatedly found that the side positioning fixation in the lying device according to the invention, which was initially designed and built by her for ultrasound examinations in the first months of life (but is not limited to this use), has an unexpectedly calming effect on children lying in it. This effect is achieved by the unusual shape of the lying device according to the invention, wherein the children experience a calming and close-fitting fixation in the area of the torso, which at the same time prevents a prone or supine position. In addition, the additional space in the head and foot region of the lying device allows unrestricted movement and at the same time the desired positioning of the child's limbs and head in the lateral position, thus providing an optimal basis from a developmental neurological point of view.

Through the combination of a large air volume in the head area and the fixing tapering of the lying device in the central region, which causes a stabilized lateral position, the lying device according to the invention can, in addition to increasing the child's well-being, also achieve the surprising effect that, on the one hand, a prone position of the child is prevented, in which there can be an increased probability of sudden infant death syndrome. Supine position of the child, which can lead to a flattening of the back of the head, can also be avoided. Sleeping in the prone position is considered a risk factor for sudden infant death syndrome in children in the first year of life. Supine position has been recommended for decades as one of the most important factors in the prevention of sudden infant death syndrome for newborns or infants in the first year of life during sleep. The supine position is considered a reliable method, as newborns and infants are generally unable to turn on their own before the age of six months, so that the recommended supine position prevents them from rolling over into the prone position. In principle, there is a risk that the infant will roll over into the prone position every time they are not in a fixed lateral position until their 1st birthday.

Unfortunately, early supine positioning also has negative consequences for some children. On the one hand, newborns and infants sleep significantly worse on their backs, as newborns and infants are unable to cope well with the sudden lack of space after 9 months of being cocooned in the embryo position in the womb. As newborns and infants lack boundaries and contact after birth, especially at the center and front of the body, regular sleep in the supine position is often disturbed by shooting muscle twitches (myoclonus) and motor reflexes. The increased restlessness promotes vortex formation in the intestines and thus flatulence. Another negative effect of the recommended supine position for infants is the flattening or deformation of the back of the head (plagiocephalus) caused by the supine position, which—once it has occurred—can only be treated by strict positioning, physiotherapy or, in severe cases, possibly unpleasant helmet therapy.

In a further preferred embodiment of the invention, the shape comprises at least one interior padding (6) which can be attached to at least one inner wall (3a) in the central region (3) in order to reduce the diameter (3b) of the central region (3).

In a further preferred embodiment of the invention, the smaller diameter of the central region (3) and/or the at least one interior padding (6) in the central region (3) are configured to prevent a prone or supine position of the child.

Since infants, newborns, toddlers and children are of different sizes and grow, in specific embodiments of the lying device, at least one additional interior padding (6) can be attached to the inside of the at least one padding insert (6) in the central region in order to adjust the diameter (3b) of the central region (several padding inserts can optionally be attached on top of each other in order to gradually reduce/increase the diameter of the central region). In this way, embodiments of the lying device can be used for infants, newborns as well as for larger toddlers, so that a lying device can be adapted to the size of the newborn as it grows. On the one hand, this has the advantage that the newborn or (small) child is optimally fixed in the lying device in the lateral position at every stage of growth without restricting the child's well-being. This flexible size adjustment also means that costs and resources are saved, as no new lying device needs to be purchased as the newborn or toddler grows.

Due to the flexible size adjustment of the central region, various embodiments for the fixation of the child are possible: e.g., embodiments 1) without adding a padding insert in the central region for larger (small) children are possible, as well as, e.g., 2) with the addition of at least one padding insert in the central region and e.g., 3) with the addition of at least one additional padding insert on the inside of an already attached padding insert (so that two padding inserts are attached on top of each other). Thus, by adding additional padding to the inner walls of the central region of the lying device, the diameter of the central region, which fixes the child's torso in the lateral position, can be gradually increased or decreased so that the lying device can be individually adapted to the size of the child. In one embodiment, the interior padding can be folded down and/or attached as individual parts.

In embodiments of the invention, the padding inserts can be fixed in the central region in various ways, wherein the padding inserts (6) can optionally be attached to the inner wall (3a) of the central region or to a padding insert (6) already attached in the central region. In embodiments, the means for fixing the interior padding are selected from the group comprising Velcro, hooks, snaps, buttons, zippers, eyelets, straps, loops, anti-slip materials (e.g., rubber), by insertion into a pocket and/or loop sewn/attached to the wall of the central region, and any combination of said means.

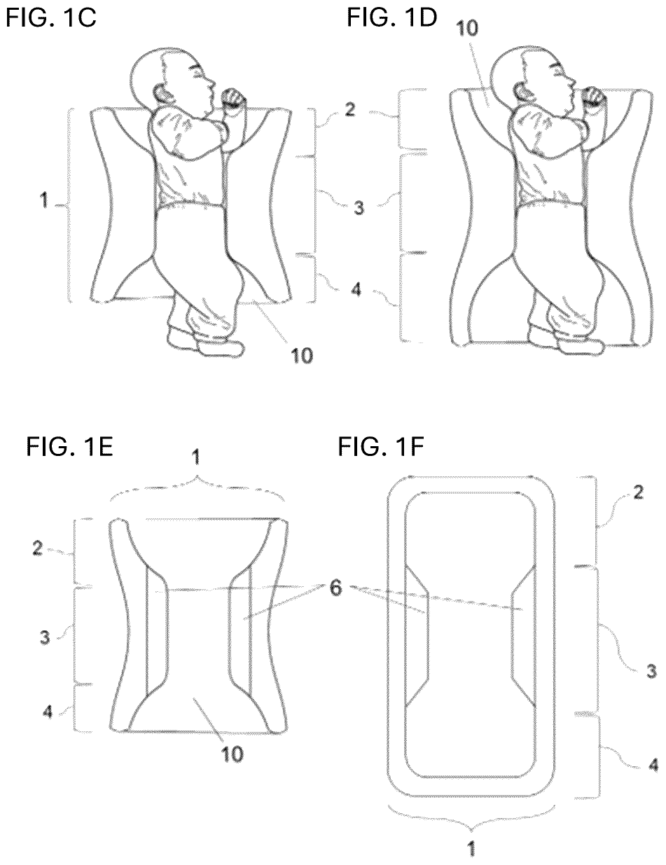

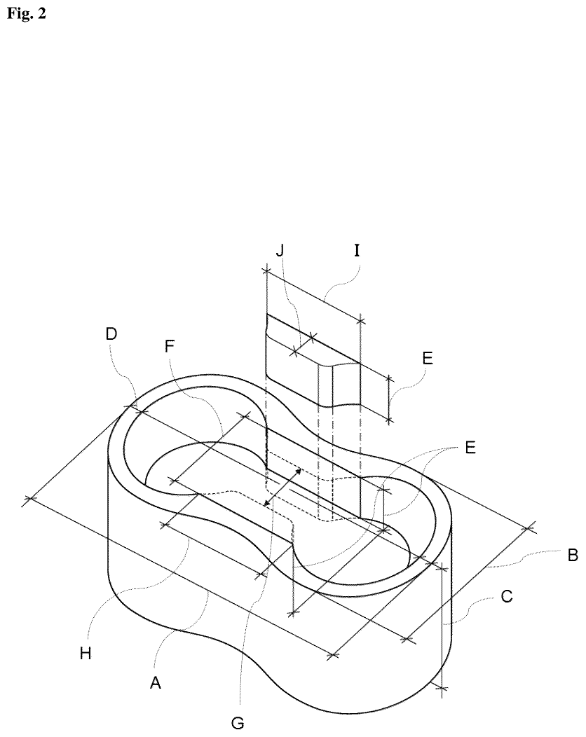

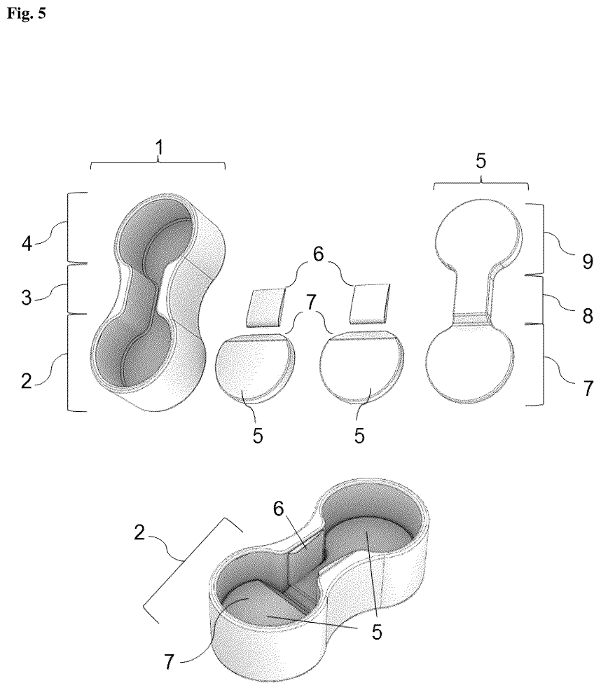

In some embodiments of the lying device (1), the at least one interior padding (6) is wedge-shaped (see, for example, FIGS. 3 and 6). In specific embodiments of the lying device, the at least one interior padding is cut straight towards the inside so that “splints” are formed which fix the torso of the child in the lateral position (see FIGS. 1B and 2). In embodiments, other shapes of the interior padding are also possible, wherein differently shaped interior padding can also be combined in the lying device. In certain embodiments, the entire construction of the lying device is made of one piece, so that the interior padding, which is responsible for fixing the child in the lateral position, is fixed and is thus itself fixed. In other embodiments, the lying device is made from more than one piece, so that individual components are replaceable and/or removable.

In embodiments, the “splint”-shaped at least one interior padding can represent and/or comprise at least one rail/padding towards the inside, which can be set or adjusted in a mechanical manner so that the inner diameter of the central region can be variably modified or reduced relative to the foot and/or head region. Mechanical adjustment mechanisms may include, for example, mechanical levers, linkages and/or joints. In embodiments, the inner diameter of the central region can be reduced by modifying the filling of at least one interior padding and/or by layering several interior paddings on top of each other.

The skilled person is familiar with various embodiments of interior padding and adjustment mechanisms, e.g. splint or splint-like (possibly mechanical) device components, which can be used for the at least one interior padding in order to variably adjust/reduce the inner diameter of the central region.

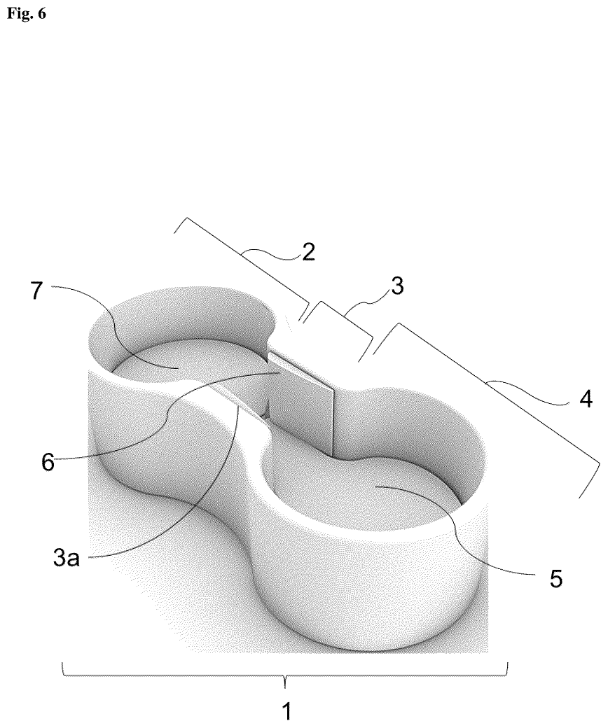

In embodiments, the smaller diameter of the central region refers to the outer dimensions of the shape, so that the outer structure of the lying device also has a smaller diameter than the head and/or foot region of the device, so that, for example, a tapered shape similar to a FIG. 8 and/or an hourglass is formed (see, for example, FIG. 1A-E). In embodiments, the smaller diameter of the central region refers to the inner dimensions of the shape, such that the diameter of the lying device is smaller (smaller inner diameter) only in the central region, as compared to the head and/or foot region of the device (see, for example, FIG. 1F). In some of these embodiments, the lying device may have an outer shape in which the outer dimensions of the central region have the same circumference or width as the head and/or foot region, with the central region only having a smaller inner diameter compared to the inner diameter of the head and/or foot region. Embodiments of this device may comprise a rectangular or nearly rectangular outer shape, wherein the inner dimensions and/or inner region of the device is “tapered” and resembles, for example, a figure eight and/or an hourglass (see, for example, FIG. 1F). In specific embodiments of this device, the inner diameter of the central region may be reduced relative to the foot and/or head region by additional interior padding or other inserts.

In embodiments of the invention, the lying device (1) comprises at least one mattress inlay (5), wherein preferably the at least one mattress inlay (5) is thicker in the head region (7) than in the central (8) and foot regions (9).

In embodiments of the lying device (1), at least one mattress (5) or mattress inlay (5) is inserted, which in preferred embodiments is slightly raised at the head in order to relieve pressure on the arm lying below in the lateral positioning of the child. In some embodiments, the mattress (5) or mattress inlay (5) has a thicker/raised head area that serves as a pillow, in other embodiments the mattress comprises a separate pillow. In various embodiments, the at least one mattress (5) or mattress inlay (5) can be designed, for example, for small babies with only a slight head elevation or for larger babies with a slightly higher head elevation. Other embodiments are also envisaged. In various embodiments, the pillow has a certain thickness. For example, pillows with a greater thickness can be used for larger children. Preferably, pillows with different thicknesses/heights can be combined with a mattress, which allows additional customization of the lying device to the child and its growth.

In a further embodiment, the mattress (5) or mattress inlay (5) has a reduced thickness in the head region (7), wherein an additional pillow (7) can preferably be used in these embodiments. In a further embodiment, the at least one mattress has a reduced thickness in the head region (7), so that an additionally used pillow is fixed in the head region (7) of the mattress (5) and the lying device and a slipping of the pillow, for example downwards, is prevented. In specific embodiments, the mattress (5) or mattress inlay (5) comprises only one pillow or head section (7).

In an alternative embodiment, the at least one mattress has a uniform thickness in the head section (7), central section (8) and foot section (9).

In a further embodiment of the lying device (1) according to the invention, the at least one mattress inlay (5) comprises a linked part or several separate or linked parts, comprising a central part (8), a head part (7) and/or a foot part (9), wherein the separate or linked parts of the mattress inlay (5) have a different or the same thickness.

In a further embodiment of the lying device (1) according to the invention, the at least one mattress inlay (5) comprises one linked part or several separate or linked regions, comprising a head region (7), a central region (8) and a foot region (9), wherein the separate or linked parts of the mattress inlay (5) have a different thickness or the same thickness.

In embodiments, a mattress inlay or mattress may be between 1 and 15 cm thick, between 1 and 10 cm thick, or between 2 and 7 cm thick. In embodiments, if the use of an additional pillow is contemplated or if the mattress has a uniform thickness, it is preferably between 2 and 5 cm thick. In embodiments, the thickness of the mattress also depends on the firmness of its material. In order to enable optimum lying comfort, mattresses made of softer materials are preferably thicker than mattresses made of firmer materials, into which the child does not sink as deeply. In some embodiments, the pillow is made of the same material as the mattress. In alternative embodiments, the pillow is made of a different material and/or a material with a different firmness than the rest of the mattress inlay.

In some embodiments, the mattress inlay is covered with a water-repellent or waterproof material or impregnation to prevent soiling of the mattress. In some embodiments, the mattress may additionally be fixed to the lying device. In some embodiments, the means for fixing the mattress are selected from the group comprising Velcro, hooks, snaps, buttons, anti-slip materials (e.g. rubber), zippers, eyelets, straps or any combination of said fixations.

In a further embodiment of the lying device (1) according to the invention, the child is preferably a newborn or an infant. In a further embodiment of the lying device (1) according to the invention, the child is a toddler under 3 years of age.

In embodiments of the lying device (1) according to the invention, the lying device (1) has outer dimensions (width×length) of 25-50×60-100 cm, preferably 30-40×65-85cm, wherein the outer walls of the lying device (1) are between 15-30 cm high, preferably between 20-35 cm high.

In embodiments of the invention, the sections or parts of the lying device (1) according to the invention have defined dimensions. The dimensions of specific embodiments of the invention are shown in FIG. 2.

The dimensions A-J defined herein are to be understood as separate and independent embodiments of the lying device according to the invention, and do not exist in any particular or inevitable combination with each other. Accordingly, in embodiments of the lying device, any specific value of one dimension may be combined with any value of other dimensions, or may exist solely for one embodiment. In other words, in specific embodiments of the lying device according to the invention, parts of the lying device (1) according to the invention have the following dimensions, wherein the dimensions of different parts are not necessarily combined with each other and may occur individually or exist or be combined independently of each other in embodiments.

A refers to the overall length of the lying device (1). In embodiments of the invention A is between 60-90 cm, preferably between 65-80 cm, in a preferred embodiment A is between 70-75 cm long.

B denotes the overall width of the lying device. In embodiments of the invention B is between 20-50 cm, preferably between 25-40 cm, in a preferred embodiment B is between 30-35 cm.

C denotes the overall height of the lying device (1). In embodiments of the invention C is between 10-40 cm, preferably between 15-35 cm, in a preferred embodiment C is between 20-30 cm high.

D indicates the width of the wall (wall thickness) of the lying device (1). In embodiments of the invention D is between 1-10 cm, preferably between 1-7 cm, in a preferred embodiment D is between 2-5 cm thick.

E refers to the height (internal height) of the wall of the lying device (1) when a mattress inlay is inserted and is measured from the top of the mattress inlay (5) of the lying device.

In certain embodiments, the at least one padding insert (6) has the same height E, so that its upper edge is flush with the upper edge of the wall of the lying device (1) when the at least one padding insert is inserted into the central region (3) of the lying device (1) and rests on the mattress inlay.

In some embodiments, the at least one interior padding (6) has a height E, which refers to a mattress inlay with a large thickness, so that the at least one interior padding does not protrude beyond the edge of the lying device even when a thick mattress is used.

In embodiments of the invention E is between 10-40 cm, preferably between 10-30 cm, in a preferred embodiment E is between 15-25 cm high.

F denotes the inner spacing (3b) of the inner walls (3a) of the central region (3) without the interior padding(s) (6) inserted. In embodiments of the invention, F is between 10-35 cm, preferably between 15-30 cm, in a preferred embodiment F is between 15-25 cm wide.

G denotes the inner spacing of the inner walls (3a) of the central region (3) with inserted interior padding(s). Accordingly, the inner distance or inner diameter of the central region G can be regulated in embodiments of the lying device by inserting or removing one or more interior padding. In embodiments of the invention, the distance of G is derived from the length of F (3b) minus the sum of the thickness of the at least one interior padding(s) (6) J. In embodiments of the invention G is between 5-25 cm, preferably between 7-20 cm, in a preferred embodiment G is between 10-15 cm wide.

H denotes the length of the inner wall (3a) of the central region (3). In embodiments of the invention H is between 5-30 cm, preferably between 10-25 cm, in a preferred embodiment H is between 12-20 cm long.

I denotes the length of the at least one interior padding, where I is preferably equal to the length H of the inner wall (3a) of the central region (3).

In a preferred embodiment, I has the same dimensions as H.

J denotes the thickness of the at least one interior padding(s) (6). In embodiments of the invention, J is between 1-15 cm, preferably between 2.5-10 cm, in a preferred embodiment J is between 3-7.5 cm thick. In some embodiments, there are interior paddings with the same and/or different thicknesses J, which can be combined with each other as required and can be or are present in the central region of the lying device. These embodiments offer versatile adjustment possibilities and particular flexibility for a child during growth, or depending, for example, on the child's clothing and/or the blanket used.

In embodiments, the base of the lying device (1) has a thickness between 1-10 cm, preferably between 2-7 cm, in a preferred embodiment the base of the lying device is between 3-6 cm thick. In specific embodiments, the thickness of the base may depend on the strength or stability of the materials used, so that in some embodiments in which a stable and/or less flexible material is used for the base, the base has a lower thickness. In some embodiments, the base comprises several different materials with different thicknesses and strengths/stabilities and/or properties.

In a specific embodiment, the parts of the lying device (1) according to the invention have the following dimensions A: 73-75 cm, B: 33-35 cm, C: 20-25 cm, D: 4-5 cm, E: 13-18 cm, F: 19-22 cm, G: 11-13 cm, H: 15-17 cm, I: 15-17 cm, J: 3-5 cm, wherein the base of the lying device is 3-5 cm thick.

In a further specific embodiment, the parts of the lying device (1) according to the invention have the following dimensions A: 74 cm, B: 34 cm, C: 20 cm, D: 3 cm, E: 14 cm, F: 20 cm, G: 12 cm, H: 16 cm, I: 16 cm, J: 4 cm, wherein the base of the lying device is 3 cm thick.

Accordingly, in a specific embodiment, the lying device according to the invention has external dimensions (A) 74 cm long and (B) 34 cm wide. The side walls of this embodiment of the lying device (C) are 20 cm high and (D) 3 cm thick. There is a 3 cm thick base at the lower edge. The lying device is open at the top so that a child can be placed in it. The “tapered” central region of the lying device is reinforced on both sides on the inside with interior padding. The two interior padding inserts are cut straight towards the inside, creating two “splints” into which the child's torso is fixed in the lateral position. The internal dimensions of this specific embodiment are 68 cm long and 28 cm wide, and 20 cm wide in the central region between the two interior paddings (G).

In embodiments of the lying device according to the invention, all figures and ranges can be understood as +/−25%, +/−20%, +/−10%, +/−5%.

In embodiments, new numerical ranges of dimensions can also be formed from the existing values, which can be routinely performed by a person skilled in the art.

In a further embodiment of the lying device (1) according to the invention, the lying device is made wholly or partly of a material selected from plastic, foam and/or natural material.

In embodiments of the invention, the materials “plastic”, “foam” and “natural material” for the manufacture of the lying device according to the invention include, but are not limited to, materials selected from the group comprising plastic, plastic, polyester, polyurethane, PET, foam, bio-plastic, natural materials, plant-based plastics, plant-based foams, wood, wood fibers, natural fibers, linen, cotton, flax, jute, hemp, silk, wool, fur, leather, artificial leather, plant-based artificial leather, velvet, velour, fleece, materials based on one or more of the enumerated materials and/or any combination of these materials. The materials are preferably predominantly pollutant-free and/or certified organic materials. The use of recycled materials, which are preferably free of harmful substances, is also considered.

In some embodiments, the base of the lying device is made of a more stable, stronger and/or thicker material than the walls so that it provides stability to the lying device. In certain embodiments, a water and/or dirt repellent or waterproof material or coating is applied to the outside of the base, which protects the underside of the lying device from soiling. In some embodiments, additional feet may also be provided to prevent direct contact of the underside of the lying device with the base.

In a further embodiment of the lying device (1) according to the invention, the lying device (1) is configured for use as a carrying bag. According to the invention, these embodiments have a central region (3), a head region (2) and a foot region (4). In these embodiments, the edge of the lying device should be continuous.

In a specific embodiment, the lying device (1) additionally has a carrying function, e.g., carrying handles, shoulder strap(s) and can thus additionally be used as a carrying or transport device or as a carrying bag for children. According to the invention, these embodiments have a central region (3), a head region (2) and a foot region (4). In these embodiments, the edge of the lying device should be continuous.

In a further embodiment, the invention comprises a (separate) bag or carrying case which encloses the lying device according to the invention, thus enabling the lying device to be portable. In some such embodiments, embodiments of the lying device comprising a central region, a head region and a foot region, as well as embodiments of the lying device without a foot and/or head region, or embodiments of the lying device having an open/interrupted/no edge at the head and/or foot region, may be used. In one embodiment of this variant, two straps each enclose the central region of the lying device and, in a specific embodiment, open into four eyelets below the edge of the bag. Carrying straps can be attached to these eyelets, so that the lying device according to the invention can be used and carried like a carrycot. Other embodiments of carrier straps are also possible.

In a further embodiment of the lying device according to the invention, the shape of the lying device (1) is configured by the smaller diameter of the central region (3) to provide a recess for the hip of the carrier when used as a carrying bag.

The “tapered” embodiment of the lying device according to the invention, which may resemble a figure eight shape or an hourglass, for example, allows the lying device to fit against the carrier's hips when worn with the “bulge” of the narrower central region. This fixes the lying device according to the invention to the carrier's hip when worn. In one embodiment, the length of optionally available carrying straps of the lying device according to the invention can additionally be adapted in such a way that the fixation on the hip of the person wearing it can be optimally adapted, which has the advantageous effect of increasing wearing comfort. In the embodiment of the lying device as a carrying bag, the lying device according to the invention combines increased sleeping comfort for children with increased carrying comfort for the person carrying the child, e.g. parents.

In a further embodiment of the lying device (1) according to the invention, the dimensions of the lying device (1) enable it to be used as a carrying bag or as a removable insert of a commercially available carrying bag, a commercially available stroller or a commercially available baby carriage.

In specific variants of this embodiment, the dimensions of the lying device are specifically adapted to the dimensions of a particular baby carriage or baby carriage.

### Optional Medical Uses

In a further aspect, the invention relates to the lying device (1) according to the invention for use in the treatment or prevention of occipital flattening (plagiocephaly) in newborns or infants. A corresponding medical application may be recommended for specific patients, e.g., for newborns or infants with occipital flattening.

In embodiments of the use according to the invention, the lying device (1) enables stable lateral positioning of a child, wherein the shape comprises a central region (3), and at least one head region (2), and/or a foot region (4), wherein the central region has a smaller diameter than the head region (2) and the foot region (4).

In embodiments of the use of the lying device according to the invention in the treatment or prevention of occipital flattening, the lying device enables stable lateral positioning of a child, wherein the shape comprises a central region (3), and at least one head region (2), and/or a foot region (4), wherein the central region has a smaller diameter than the head region (2) and the foot region (4), and wherein the shape preferably includes at least one interior padding (6) which can be attached to at least one inner wall (3a) in the central region (3) to reduce the diameter (3b) of the central region (3). In embodiments of the use according to the invention, the lying device (1) enables stable lateral positioning of a child, wherein the shape comprises a head region (2), a central region (3) and a foot region (4), wherein the central region has a smaller diameter than the head region (2) and the foot region (4).

In embodiments of use according to the invention, the lying device comprises at least one interior padding (6) which can be attached to at least one inner wall (3a) in the central region (3) in order to reduce the diameter (3b) of the central region (3).

In embodiments of use, the lying device comprises at least one mattress inlay (5), wherein preferably the at least one mattress inlay (5) is thicker in the head region (7) than in the central (8) and foot regions (9) or wherein the mattress inlay (5) has a uniform thickness and comprises a separate cushion.

In embodiments of the use of the lying device (1) according to the invention, the smaller diameter of the central region (3) and/or the at least one interior padding (6) in the central region (3) are configured to prevent a prone or supine position of the child.

In embodiments of the use of the lying device (1) according to the invention, the child is preferably a newborn or an infant.

As previously described herein, the lying device according to the invention achieves the surprising effect that a stabilized lateral position is brought about by the combination of a large volume of air in the head region and the fixing tapering of the lying device in the central region. In specific embodiments, the lying device according to the invention can also be used in a medical, preventive and/or therapeutic context. Particularly in this context, in embodiments, the combination of a large air volume in the head region and the fixing tapering of the lying device in the central region, which causes a stabilized lateral position, can achieve the surprising effect, in addition to the increased well-being of the child, that the probability of sudden infant death syndrome is reduced by a prone position of the child, while at the same time a flattening of the back of the head is prevented by a supine position of the child. This effect has a great advantage over the usual supine position or subsequent helmet therapy, especially when using the present invention in the treatment or prevention of occipital flattening, since on the one hand, in the case of an already existing plagiocephaly, a painful and severely restrictive helmet therapy for children can be avoided by means of a positioning therapy using the lying device according to the invention. On the other hand, the use of the lying device according to the invention can successfully prevent the flattening of the back of the head caused by back positioning right from the birth of the child and thus also avoid the later consequences of plagiocephaly.

Thus, the present lying device according to the invention represents a significant improvement over the prior art, as it provides a safe, comfortable and uncomplicated way of lateral positioning of children from birth, which was not possible with the means available up to now.

### DETAILED DESCRIPTION OF THE INVENTION

All cited patent and non-patent literature documents are hereby incorporated by reference into the present disclosure in their entirety.

The present invention relates to a lying device which, due to its shape, enables stable lateral positioning of a child.

In the context of the present invention, the term “child” includes the following age groups: Newborns up to the completed 28th day of life, infants from the beginning of the 29th day of life up to the completed 12th month of life, toddlers from the beginning of the 2nd to the completed 3rd year of life and/or children from the beginning of the 4th to the completed 12th year of life. Preferably, the term child refers to children up to the age of 4 years who have a maximum height/length of 90 cm. In general, in the context of the present invention, the term “child” can be used as a generic term for all persons under the age of 12, preferably under the age of 4. In embodiments, the term “infant” includes all children from birth to 12 months of age.

According to the invention, the embodiments described herein, which relate to children, also relate to infants and/or newborns and vice versa. In the same way, embodiments which are described for infants also relate to newborns and vice versa.

Sudden infant death syndrome (SIDS) is the term used to describe the unexpected, sudden death of a newborn or infant before the 365th day of life. Despite a thorough examination, the cause of death cannot usually be clarified or cannot be clearly determined. Children usually die in their sleep at night—boys more often than girls. The majority of SIDS victims are found by their parents in the prone position, so sleeping in the prone position is associated with an increased risk of SIDS. Accordingly, doctors recommend sleeping in the supine position for children in the first year of life, as they are not yet able to turn independently, so that a supine position prevents them from rolling over into the prone position. Although preferred by many children for sleeping, the side sleeping position is not recommended as it cannot be held stable by newborns and infants, so there is a risk of rolling into the risk-associated prone position.

“Plagiocephalus” or “plagiocephaly” is an asymmetrical flattening of the back of the head, wherein the ratio of length to width of the skull is not altered. Prolonged lying in the supine position can affect the shape of the still malleable skull of infants at the back of the head and lead to plagiocephaly. The brain is not affected by the shape of the skull. Premature suture closure can also lead to facial asymmetries. The supine position recommended by doctors for infants leads to a significant reduction in sudden infant death syndrome, but also favors position-related head deformation.

The terms mattress inlay and mattress are interchangeable in the context of the present invention. In embodiments of the present invention, a mattress or mattress inlay may also comprise a pillow.

In the context of the invention, an “interior padding”, which is suitable for reducing the diameter of the central region compared to that of the head and/or foot region, may be made of a firm and/or flexible material, wherein in embodiments, interior padding may also comprise, for example, an air, water, granulate, foam, fabric, plastic, natural material, gel filling or similar and/or usable fillings. In general, interior padding can consist of one or more materials and/or layers.

In specific embodiments, the interior padding comprises an adjustment device and/or an adjustment mechanism, e.g. mechanical means (comprising e.g. levers, linkages, joints, etc.) or the possibility of letting filling in and out. Any design of interior padding is conceivable which enables the function of reducing the diameter of the central region in accordance with the invention. Accordingly, various forms of interior padding are also possible, with non-limiting examples of embodiments of straight, wedge-shaped, rounded, ergonomically shaped and/or angular forms of interior padding being conceivable.

### FIGURES

The invention is further specified by the following figures. These are not intended to limit the scope of the invention, but represent preferred aspects of the invention disclosed to further illustrate the invention described.

### BRIEF DESCRIPTION OF THE FIGURES

FIG. 1: Schematic of the lying device according to the invention with the child lying in it in the lateral position (A) and with attached padding in the central region (B), (C-E) Schematic of the lying device “open at the top and bottom”, which resembles an hourglass open at the top and bottom with the child lying in it in the lateral position (C, D) and with attached padding in the central region (E). (F) shows an embodiment in which the reduced diameter in the central region is only present in the interior of the device.

FIG. 2: Dimensions of a specific embodiment of the invention.

FIG. 3: Top view of various embodiments of the lying device according to the invention.

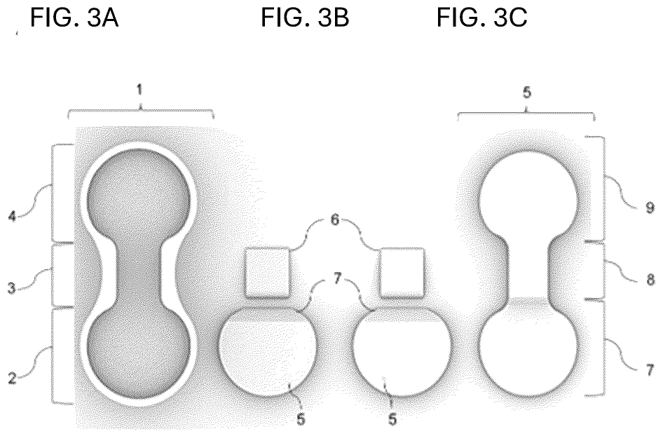

FIG. 4: Top view of various embodiments of the lying device according to the invention. Comparative view of the lying device with and without inserted mattress.

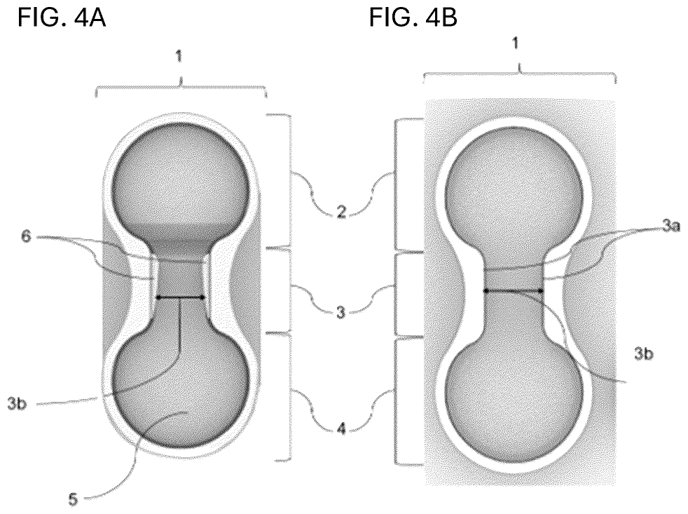

FIG. 5: 3-dimensional representation of the embodiments of the lying device (1) and the mattress (5), pillow (7/5) and interior padding (6) shown in FIGS. 3 and 4.

FIG. 6: 3-dimensional representation of the lying device (1) shown in FIGS. 3 and 4 with an inserted mattress (5), an additional pillow (7) inserted in the head area (2) and two interior paddings (6) attached in the central region (3).

### DETAILED DESCRIPTION OF THE FIGURES

FIG. 1: Schematic of exemplary embodiments of the lying device (1) according to the invention with A) a child lying therein in the lateral position and B) with attached interior padding in the central region in top view. Figure A) shows the head (2), central (3) and foot regions (4) of the lying device (1). As shown in A), the toddler lies with the torso in the narrowed central region (3), wherein the child's lateral position is stabilized and the child experiences a feeling of resistance and stabilization in the torso area. This feeling of stabilization/support together with lying in the lateral position can have a calming and sleep-promoting effect on the child. It also effectively prevents prone and supine positions and the associated risks and disadvantages. The stabilizing boundary in the central region (3) can be improved and individually adapted to the child by one or more interior paddings (6), which can be attached in the central region (3), see B). The proportions of the lying device according to the invention and especially of the central region may deviate from the illustration in embodiments of the invention. Figures (C-E) show an exemplary diagram of the lying device “open” at the top and bottom, which resembles an hourglass open at the top and bottom with the child lying in it in the lateral position (C, D) and with attached interior padding in the central region (E). The base (10) is schematically indicated in the three figures (C-E). F) shows an embodiment of the invention in which the diameter in the central region, compared to the head and foot regions, is reduced solely by a narrowing/constriction in the inner region of the lateral walls of the device. For example, interior padding(s) can be used to narrow the inner diameter of the central region. In this embodiment, the outer side walls are straight (parallel) and the outer shape of the device—in contrast to the interior—has no “taper”.

FIG. 2: Dimensions of a specific embodiment of the invention. A refers to the overall length of the lying device (1), B refers to the overall width and C refers to the overall height of the lying device (1). D indicates the width of the wall (wall thickness) of the lying device (1) and F indicates the inner spacing (3b) of the inner walls (3a) without the interior padding(s) (6) inserted. E refers to the height (interior height) of the wall of the lying device (1) when measured from the top of the mattress inlay (5) of the lying device, wherein the at least one padding insert (6) preferably has the same height E, so that its upper edge is flush with the upper edge of the wall of the lying device (1) when the at least one padding insert is inserted into the central region (3) of the lying device (1). G denotes the inner distance of the inner walls (3a) with inserted interior padding(s). The distance of G is derived from the length of F (3b) minus the sum of the thickness of the at least one interior padding(s) (6) J. I denotes the length of the at least one interior padding, where I is preferably equal to the length H of the inner wall (3a) of the central region (3).

FIG. 3: Top view of various embodiments of the lying device according to the invention. A) shows the empty lying device (1) with head (2), central (3) and foot regions (4) without mattress, pillow or interior padding. B) shows embodiments of the head cushion in different material thicknesses (7/5) as well as wedge-shaped embodiments of the interior padding (6). C) shows a mattress inlay (5) comprising a head region (7), a central region (8) and a foot region (9), wherein the head region (7) has a different thickness than the central region (8) and the foot region (9).

FIG. 4: Top view of various embodiments of the lying device according to the invention. Comparative view of the B) empty lying device (1) with head (2), central (3) and foot regions (4) and A) of the lying device (1) with inserted mattress (5). Figure A) illustrates how the mattress, pillow and interior padding shown as examples in FIGS. 3B and C can be inserted into the lying device (1). The diameter (3b) of the central region (3) can be reduced by attaching interior padding (6) to the inner walls (3a) (see left-hand figure). An additional pillow inserted in the head section (2) or a mattress (5) which is thicker in the head section (7) than in the foot (8) and central region (9) can increase the child's comfort, as the head is stabilized in the lateral position and the neck and lower arm are relieved.

FIG. 5: 3-dimensional representation of the embodiments of the lying device (1) and the mattress (5), pillow (7/5) and interior padding (6) shown in FIGS. 3 and 4.

FIG. 6: 3-dimensional representation of the lying device (1) shown in FIGS. 3 and 4 with inserted mattress (5), additional pillow (7) inserted in the head region (2) and two interior paddings (6) attached to the inner wall (3a) of the central region (3).

### EXAMPLES

The invention is further characterized by the examples. These are not intended to limit the scope of the invention, but represent preferred embodiments of aspects of the invention provided to better illustrate the invention described herein.

An exemplary embodiment of the invention is shown in FIGS. 1-6.

In one embodiment example, the dimensions labeled in FIG. 2 are as follows:

The lying device (1) has the following external dimensions: It is (A) 74 cm long and (B) 34 cm wide. The side walls of this embodiment of the lying device (C) are 20 cm high and (D) 3 cm thick. There is a 3 cm thick base at the lower edge. The lying device is open at the top so that a child can be placed in it. The “tapered” central region of the lying device is reinforced on the inside on both sides by an interior padding (6). The two interior paddings (6) are cut straight towards the inside, creating two stabilizing “splints” that fix the child's torso in the lateral position. The internal dimensions of this specific embodiment are 68 cm long and 28 cm wide, and 20 cm wide in the central region between the two interior paddings (3b, G). The other dimensions for the height of the interior padding (6) (E) are 14 cm. The central region (3) is 20 cm wide/wide on the inside without padding (F) and 12 cm wide/wide with padding (G), wherein the central region (3) without padding (H) is 16 cm long. The interior padding (6) also has a length of (I): 16 cm at the outer edge with which it is fixed to the central region (3a). The thickness of the interior padding (6) is (J): 4 cm.

LIST OF REFERENCE SYMBOLS
Lying device
Head region
Central region
Foot region
Mattress
Interior padding
Head region of the mattress
Central region of the mattress
Foot region of the mattress
Base
Overall length (outer length) of the lying device
Overall width (outer width) of the lying device
Overall height (external height) of the lying device
Thickness of the wall (wall thickness) of the lying device
Height (interior height) of the wall of the lying device starting from the top of the mattress inlay of the lying device, optionally also height of an interior padding
Inner distance of the inner walls without inserted interior padding(s)/inner diameter of the central region
Inner spacing of the inner walls with inserted interior padding(s)/Inner diameter of the central region with inserted interior padding(s)
Length of the inner wall of the central region
Length of at least one interior padding
Thickness of the interior padding(s)

1. A lying device (1), which, due to its shape, enables stable lateral positioning of a child, wherein
the shape comprises a central region (3), and a head region (2), a foot region (4), or both the head region (2) and the foot region (4) wherein the central region has a smaller diameter than the head region (2) and the foot region (4).

2. The lying device (1) according to claim 1, wherein the shape includes at least one interior padding (6), which can be attached to at least one inner wall (3a) in the central region (3) to reduce the diameter (3b) of the central region (3).

3. The lying device (1) according to claim 1, wherein the smaller diameter of the central region (3) and/or the at least one interior padding (6) in the central region (3) are configured to prevent a prone or supine position of the child.

4. The lying device (1) according to claim 1, wherein the lying device (1) further comprises at least one mattress inlay (5).

5. The lying device (1) according to claim 1, wherein the lying device is made wholly or partly of a material selected from the group consisting of plastic, foam, natural material, and a combination thereof.

6. The lying device (1) according to claim 1, wherein the child is a newborn or an infant.

7. The lying device (1) according to claim 4, wherein the at least one mattress inlay (5) comprises one linked part, or several separate or linked parts, the at least one mattress inlay (5) defining a central part (8), and at least one head part (7), and/or a foot part (9).

8. The lying device (1) according to claim 1, wherein the shape comprises a central region (3), a head region (2), and a foot region (4).

9. The lying device (1) according to claim 8, wherein the lying device (1) has the dimensions (width×length) of 25-50×60-100 cm, wherein the outer walls of the lying device (1) are between 15-30 cm high.

10. The lying device (1) according to claim 8, wherein the lying device (1) is configured for use as a carrying bag.

11. The lying device (1) according to claim 10, wherein the shape of the lying device (1) is configured by the smaller diameter of the central region (3) to provide a recess for the hip of the carrier when used as a carrying bag.

12. The lying device (1) according to claim 1, wherein the dimensions of the lying device (1) allow it to be used as a carrying bag or as a removable insert of a commercially available carrying bag, a commercially available stroller or a commercially available baby carriage.

13. The lying device (1) according to claim 1, wherein the edge of the lying device has a different height in the central region (3), head region (2) and/or foot region (4) or is interrupted in the head and/or foot region.

14. The lying device (1) according to claim 7, wherein the separate or linked parts of the mattress inlay (5) have a different or the same thickness.

15. The lying device (1) according to claim 8, wherein the lying device (1) has the dimensions (width×length) of 30-40×65-85 cm, and wherein the outer walls of the lying device (1) are between 20-35 cm high.

16. A method of treating a newborn or infant subject comprising using the lying device according to claim 1 for the treatment or prevention of occipital flattening (plagiocephaly) in a newborn or an infant subject.

17. The method according to claim 16, wherein the smaller diameter of the central region (3) of the lying device is configured to prevent a prone or supine position of the newborn or infant subject.

18. The method according to claim 16, wherein the lying device comprises at least one interior padding (6) which can be attached to at least one inner wall (3a) in the central region (3) in order to reduce the diameter (3b) of the central region (3) to a smaller diameter.

19. The method according to claim 17, wherein the smaller diameter of the central region (3) and the at least one interior padding (6) in the central region (3) of the lying device are configured to prevent a prone or supine position of the newborn or infant subject.
